(19) United States
(12) Patent Application Publication
(10) Pub. No.: US 2019/0038115 A1
(43) Pub. Date: Feb. 7, 2019

(54) DEVICE FOR REACHING PAST THE NECK OF THE UTERUS OF A LIVESTOCK ANIMAL FOR THE PURPOSE OF TRANSFERRING MATERIAL OR SUBSTANCE WITH A REPRODUCTVE, THERAPEUTIC OR DIAGNOSTIC PURPOSE OR FOR COLLECTING SAMPLES FROM THE UTERUS

(71) Applicant: ELEXINN, Migennes (FR)

(72) Inventors: Agathe DECHERF, Migennes (FR); Pierrick DREVILLON, Migennes (FR)

(21) Appl. No.: 16/073,591

(22) PCT Filed: Jan. 30, 2017
(86) PCT No.: PCT/FR2017/050211

§ 371 Date: Jul. 27, 2018

(30) Foreign Application Priority Data
Jan. 29, 2016 (FR) 1650764

(51) Int. Cl. A61B 1/015; A61B 10/02; A61D 7/00; A61D 19/02; A61D 19/04; A61M 31/00; A61B 1/303; A61B 1/00; A61B 1/05; A61B 1/06

(52) U.S. Cl. CPC A61B 1/015; A61M 2250/00; A61D 7/00; A61D 19/027; A61D 19/04; A61M 31/005; A61B 1/303; A61B 1/00154; A61B 1/00016; A61B 1/00052; A61B 1/00089; A61B 1/05; A61B 1/0676; A61B 1/0684; A61B 1/00101; A61B 1/00094; A61B 1/00066; A61B 2503/40; A61M 2210/1433; A61B 10/0291

(57) ABSTRACT

Device for vaginal access to the uterus of a livestock animal for transfer of material or collecting samples, includes a body integrally connected to gripping element, a first passage extending through the device and opening out at two ends in order to establish an access route between the inside and the outside of the animal, the body being straight and rigid in order to permit introduction into the vagina, and a cup being provided at its distal end, a projection extending from the bottom of the cup and having a through-hole in communication with the first passage, the cup having an orifice in communication with a second passage extending through the body and connected to suction element, to place the cup flat against the fornix of the neck of the uterus, with viewing element likewise being provided in order to permit viewing at the front of the cup.

The present invention addresses the general problem of accessing, in vivo, the uterus of a livestock animal, for the purpose of an atraumatic transfer of material or substance with a reproductive, therapeutic or diagnostic purpose, or for the purpose of a sampling from the uterus, and in particular it relates to a device enabling such an access via the vagina.

The expression “material or substance with a reproductive purpose” (or reproduction) refers to semen or an embryo, wherein in such a case the transfer device is an artificial insemination device or an embryo transfer device. The reproduction material could be contained within a straw, as well known in the art.

The expression “material or substance with a therapeutic purpose” refers to any agent aiming to make the subject pass from a pathological state to a normal state, or to prevent a pathological state. Thus, deep intrauterine antibiotics, antiseptic solutions or isotonic wash solutions could be mentioned as examples.

The expression “material or substance with a diagnostic purpose” refers to any agent allowing or helping a physician to establish a diagnostic. Thus, marking substances such as methylene blue could be mentioned as examples.

The expression “sampling from the uterus” refers to any sampling of material or substance from the uterus, in order to carry out analyses such as the cytology, a biopsy, etc.

The present invention applies to livestock animals, in particular bovines, goats, and more generally to ungulates.

If the example of bovines is taken, in order to access the uterus of a bovine for the purpose of a transfer of material or substance with a reproductive, therapeutic or diagnostic purpose, or for the purpose of a sampling from the uterus, it is currently generally known to pass through the transrectal route.

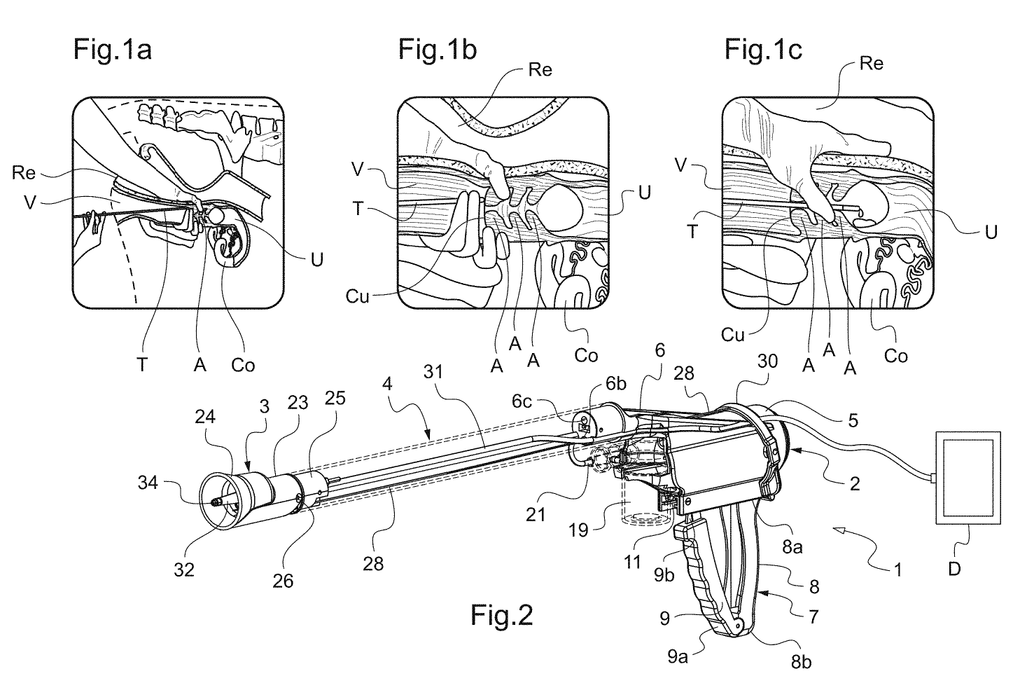

This method, illustrated in FIGS. 1a to 1c, consists, for an operator, in passing one of his arms into the rectum Re of the bovine, in order to carry out catheterization of the uterine cervix, the catheterization consisting in grasping the uterine cervix Cu and the uterine horns Co, and to align the uterine cervix Cu with a transfer or sampling catheter T held by the operator in his other hand, the catheter T being first inserted into the vagina V and then into the uterine cervix Cu so that the distal end thereof is located in the uterus U of the animal.

Indeed, the vagina V comprises, at the front part thereof, many folds to be passed in order to reach the uterine cervix Cu. The uterine cervix comprises a central channel, referred to as the cervical canal, which is generally composed of three cervical rings A that have to be aligned with one another in order to be able to insert the transfer or sampling catheter T into the uterus U of the bovine.

However, this method has several disadvantages.

It is complex and difficult to carry out, because it requires that the operator manipulates the catheter while having one of his arms inside the rectum of the animal. Thus, it can be difficult for the operator both to manipulate the internal organs of the animal with one hand, and manipulate the catheter with the other hand. In particular, the difficulty lies in crossing the cervix through blind manipulation, that is to say without viewing the organs of the animal, the operator only relying on his sense of touch.

Due to these complex manipulations, an inseminator carrying out a transrectal manipulation may suffer from musculoskeletal disorders due in particular to injuries at the shoulders.

This method further poses a risk of injury to the bovine, generally by transrectal laceration or perforation due to a too sudden manipulation of the genitalia by transrectal route, or else by damaging the walls of the vagina and/or uterus with the transfer or sampling catheter, which may in some cases lead to contamination by introduction into the uterus of substances from the vagina, and runs a risk of infection for the bovine.

Indeed, it is necessary, when using any device for the transfer of material or substance with a reproductive, therapeutic or diagnostic purpose, not to injure the animal. For example in the case of artificial insemination, the trauma associated with an injury will give rise to a local proinflammatory reaction that disrupts the phenomenon of ovulation and then of implantation of the embryo, which will limit the chances of success of the fertilization. It is therefore necessary to prevent the device from being likely, by the dimensions thereof and/or the operation thereof, to injure the animal. In other words, the device has to be atraumatic.

For the above reasons, the transrectal method requires a certain experience and is therefore currently done generally by specialized inseminator, and remains too complex to be carried out by the stock farmers themselves.

The present invention aims to solve the problems encountered with conventional transfer or sampling techniques described above by providing a device for accessing, via the vagina, the uterus of a female livestock female, for the purpose of a transfer of material or substance to the uterus or of a sampling from the uterus, and to carry out said transfer or sampling, the device avoiding the passage through the transrectal route, protecting the end of the transfer or sampling catheter, being atraumatic to the animal, and avoiding the appearance of musculoskeletal disorders for the inseminator.

The present invention therefore relates to a device for accessing, via the vagina, the uterus of a female livestock animal, such as a bovine, for the purpose of a transfer of material or substance with a reproductive, therapeutic or diagnostic purpose, or for the purpose of a sampling from the uterus, and for carrying out said transfer or sampling, the device comprising a body assembly, which is integral, at the proximal end thereof, with grasping means for an operator and has a distal end, a first passage extending through the device and opening out at the proximal and distal ends, so as to establish an access route between the inside and the outside of the animal, so as to allow the transfer of material or substance with a reproductive, therapeutic or diagnostic purpose, or the sampling, the device being characterized in that the body assembly is substantially straight and rigid so as to allow it to be inserted into the vagina of the animal, and in that is provided, at the distal end, a cup, the opening of which is oriented away from the proximal end of the body assembly, a central projection extending from the bottom of the cup and having a through hole, an end of which is in communication with the first through passage of the body assembly, the cup also having an orifice in communication with a distal end of a second through passage provided in the body assembly and the other, proximal, end of which being configured for connection to suction means which are configured to create in the cup a negative pressure that presses the cup against the fornix of the animal, the first passage then constituting an access route to the uterus from the outside of the animal, the cup also comprising a second hole adapted to receive viewing means having a field of view oriented in the distal direction of the device to allow an operator to view the surroundings ahead of the cup.

The device according to the present invention thus creates a passage for a member for transfer or sampling in the uterus of the animal, allowing the operator to very easily secure the uterine cervix to the device thanks to the connection established between the cup and the fornix and thanks to the viewing means.

The orifice in communication with the second through passage is, preferably, located in the bottom of the cup.

According to a particular embodiment, tehe viewing means comprise a camera having an objective received in the second hole, a lighting source and transmission means for transmitting images captured by the camera to a viewing device having a screen allowing the operator to observe said captured images.

The camera may be an endoscopic camera in which the lighting source is integrated, said transmission means being formed by the body of the endoscopic camera, said body extending along the body assembly to the proximal end region of the latter, where the endoscopic camera is connected to the viewing device.

Alternatively, said transmission means consist in at least one electronic circuit housed in the body assembly and connected to the camera, the at least one electronic circuit being configured to wirelessly transmit to the viewing device the images captured by the camera. Advantageously, the cup may be transparent or translucent and comprise, on the rear part thereof, light-emitting diodes arranged so as to illuminate in the distal direction of the device, the light beam of the diodes going through the transparent cup and the diodes thus constituting the lighting source for the camera.

Preferably, the cup is removably connected to the body assembly. If he wishes, the operator will be able to change the cup once the previous is soiled. The operator may also choose not to change cups between animals, in which case a sanitary protection such as the jacket described below should be used.

Preferably, to make the cup change particularly easy and fast, the cup is removably connected to the body assembly by means of the quick-release attachment type, such as reversible snap-fit attachment means.

According to a preferred embodiment, the device according to the present invention comprises an attachment piece, which is integral with the body assembly and comprises a distal region extending from the body assembly, the cup having, on the proximal side, a recess configured to receive said distal region, either one, or both, of the cup and said distal region having the means of the quick-releasing attachment type, the attachment piece further having a through passage in communication with the first through passage of the body assembly and with the through hole of the projection of the cup when the latter is attached to the attachment piece.

As indicated above, the viewing means may comprise a camera having an objective received in the second hole of the cup, a lighting source and transmission means for transmitting images captured by the camera to a viewing device having a screen. The viewing means may be an endoscopic camera as described above. According to an advantageous variant, said transmission means consist in at least one electronic circuit housed in the body assembly and connected to the camera, the at least one electronic circuit being configured so as to wirelessly transmit to the viewing device the images captured by the camera, the attachment piece and the cup being transparent, a lighting source, such as light-emitting diodes, being mounted inside the attachment piece so as to illuminate in the distal direction of the device.

According to a particular embodiment, the body assembly is of cylindrical shape and comprises a body part which is integral with the grasping means, the body part being preferably made of plastic material such as polyvinyl chloride.

The orifice of the projection of the cup may be closed by a cap, made of elastomeric material, which is integral with the projection, the cap protecting the transfer or sampling member with respect to potential secretions from the animal after removal of the plug, the cap having a central cross slit allowing a transfer or sampling member to go through it when said member is pushed by an operator, and thus to go beyond the projection

Thus, the transfer or sampling member is further protected from vaginal secretions from the bovine.

The grasping means may comprise a handle and a body, the grasping means having, in combination with the body assembly, the shape of a gun. The gun shape allows the operator to easily grasp the device.

The suction means may comprise a manual pump having a plunger and a chamber, a part of the handle of the grasping means being pivotably mounted with respect to the body of the grasping means and being connected to the plunger, whereby a movement of the handle actuates the plunger and creates a suction in the chamber.

Thus, by operating the handle several times in succession, an operator can create a negative pressure that is sufficient to press the cup against the cervix. In addition, the suction means being integrated in the device, it is not necessary to use additional suction means.

Alternatively, the suction means may be separate from the device, and for example be formed by a manually actuated, or foot actuated, vacuum pump.

According to a particular embodiment, the body assembly is of a cylindrical shape, and comprises a body part which is integral with the grasping means and a head part constituting the distal end of the body assembly, the head part being connected to the body part by a pivot connection along a diameter of the body assembly, the device further comprising actuating means which orient the head part between a so-called straight position in which the body part and the head part are aligned on a same straight line, and a so-called tilted position in which the body part and the head part form a non-zero angle to one another, preferably an angle in the range between 0° and 35°.

The entrance to the uterine cervix is usually not aligned with the vagina of the animal. In the prior art, the operator passed through the transrectal route in order to manipulate the organs and align them. In the case of the present device, tilting the distal part avoids the transrectal manipulation when the organs are not aligned.

Preferably, the actuating means orienting the head part comprise a yoke, a wire and a spring, the yoke being pivotably mounted on and with respect to the grasping means, the wire connecting the yoke and a first connection point provided in the head part, and the spring connecting the head part and a second point provided in the body part, the first and second points being located on either side of an imaginary plane which includes the pivot connection and divides the body part longitudinally into two equal parts, such that the head part is brought to the titled position when the yoke is pivoted toward the proximal end of the device, by pulling on the wire, and returns to the straight position under the action of the spring when the yoke is released.

Thus, the actuating means can be manipulated with one hand by the operator, said hand holding the device.

The distal end of the cup may be selectively closed by a plug of elastomeric material, capable of being secured to the cup by force insertion, the plug protecting the animal, during insertion, from the edges of the cup that can traumatize the animal, and protecting the inside of the cup from possible secretions of the animal, the plug having on the outer face thereof a through hole with which a thread is secured to the plug, the thread being of sufficient length so that it extends outside the animal after insertion of the device into the vagina of the animal, and the thread allowing, by pulling on it, the removal of the plug when necessary, in order to clear the passage for the transfer or sampling member.

The atraumatic feature is provided by the plug, because the shape thereof, coupled to that of the cup and the rest of the device, allows insertion into the vagina without risking damaging the walls of the vagina.

In practice, it may happen that the field of view of the viewing means is obstructed by secretions of larger dimensions than the orifice configured to be connected to the suction means for pressing the cup against the fornix of the animal. Thus, for example, phlegms, in particular phlegms during the heat period in the case of artificial insemination, are often encountered. To overcome this difficulty, according to the present invention, the cup may advantageously comprise a phlegm suction through hole, a distal end of which opens on the opening side of the cup and a proximal end of which is configured to be connected to the distal end of a suction duct, the proximal end of which is configured to be connected to phlegm suction means which are in turn configured to create a negative pressure for sucking the potential phlegm from the opening of the cup to the outside of the device via the phlegm suction through hole and the suction sheath.

The phlegm suction means can take any suitable form, such as for example means separate from the device, such as a manually actuated, or foot actuated, vacuum pump.

Said suction duct may extend inside or along the body part of the body assembly, and thus, according to the present invention, is considered to be part of the body assembly.

The present invention also relates to a cup for a device as defined above.

The cup can also comprise a sanitary jacket, which is substantially tubular and closed at the distal end thereof, rolled up on itself at the proximal end thereof, and arranged so as to envelop the cup and be able to be rolled out on the body assembly of a device as defined above, once the cup has been attached to said device.

In order to better illustrate the subject-matter of the present invention, particular embodiments will now be described, for information and non-limiting purposes, with reference to the appended drawings.

In these drawings:

FIGS. 1a to 1c are illustrations of the various steps of the method for inserting, through a transrectal route, a transfer or sampling member into the uterus of a bovine, according to the prior art;

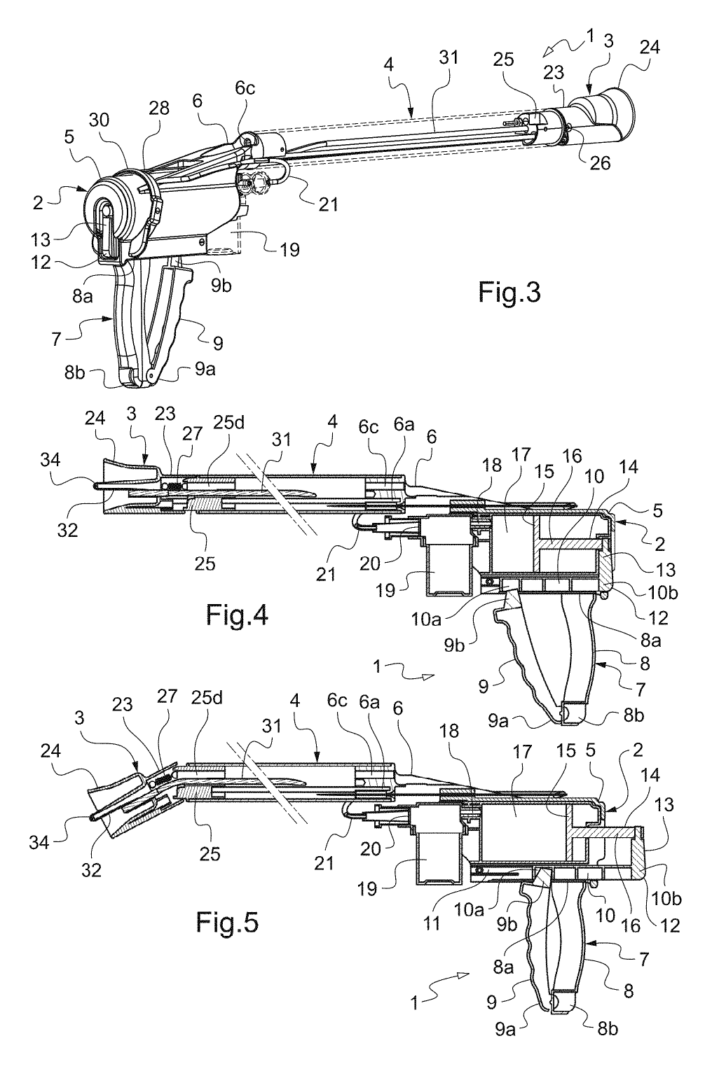

FIGS. 2 and 3 are perspective front and rear views, respectively, of the device according to a first embodiment of the present invention;

FIGS. 4 and 5 are cross-sectional views of the device according to the first embodiment of the present invention, in the straight position and in the tilted position, respectively;

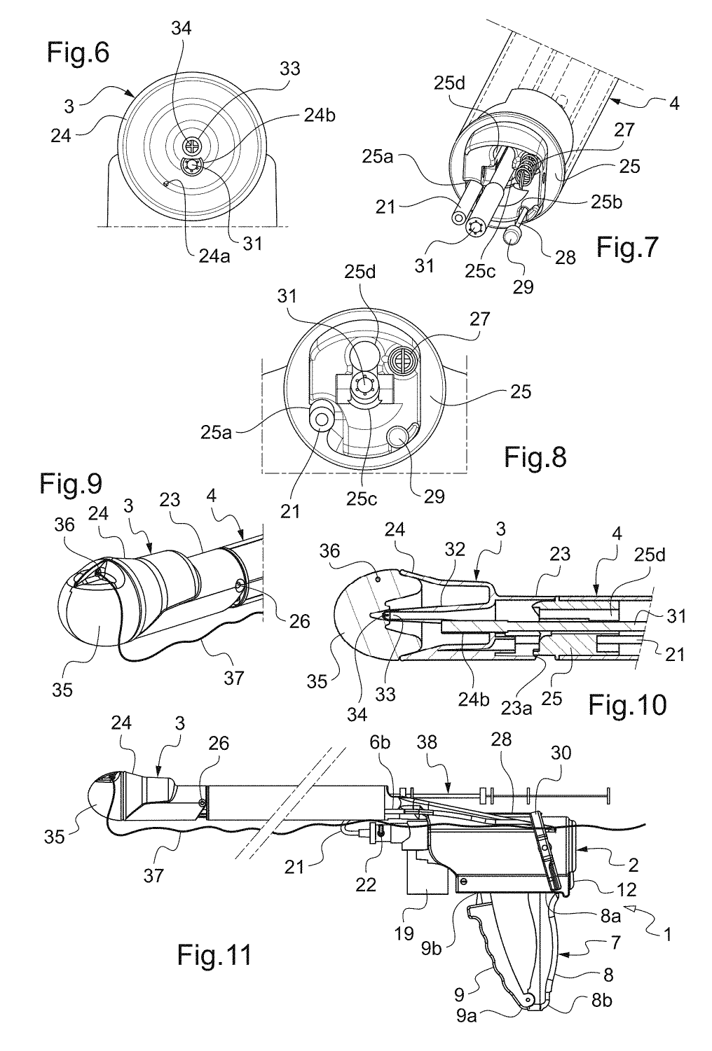

FIG. 6 is a view of the inside of the cup of the device according to the first embodiment of the present invention;

FIGS. 7 and 8 are perspective and front detail views, respectively, of the joining piece of the device according to the first embodiment of the present invention;

FIGS. 9 and 10 are perspective and side cross-sectional detail views, respectively, of the distal end of the device according to the present invention, with the plug mounted at said distal end;

FIG. 11 is a side view of the device according to the first embodiment of the present invention, in its initial position before insertion into the vagina of a bovine;

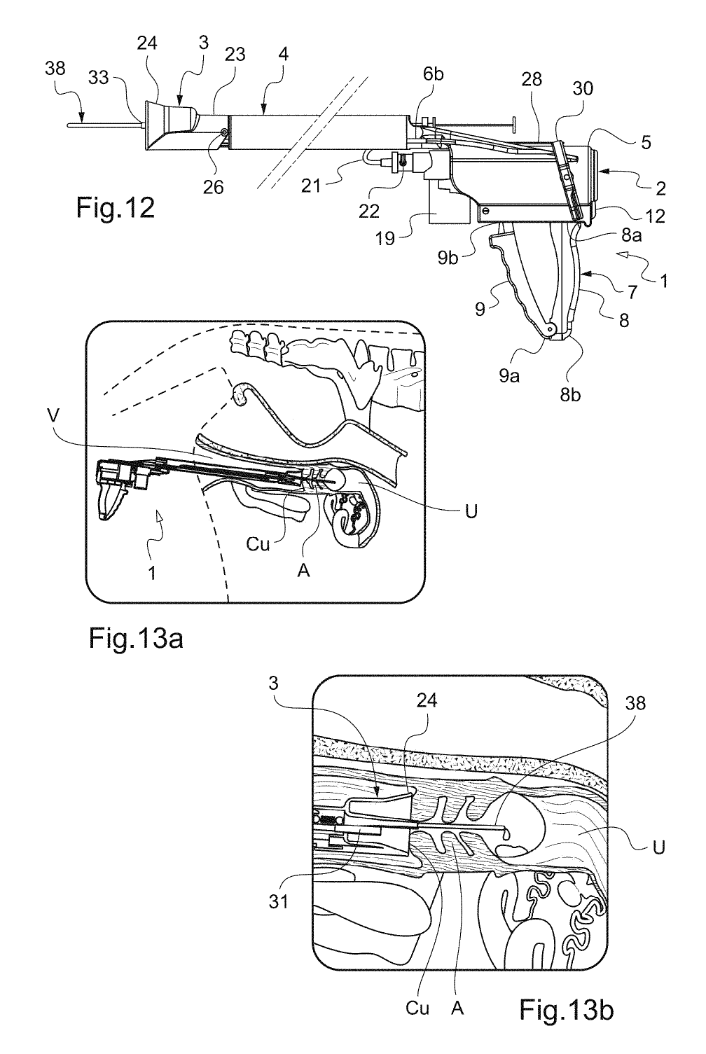

FIG. 12 is a side view of the device according to the first embodiment of the present invention, in its position for transfer of material or substance or for sampling from the uterus;

FIG. 13a is a sectional view of the device according to the first embodiment of the present invention, inserted into the vagina of a bovine, with the cup being pressed against the uterine cervix and the distal end of the transfer and sampling member being located in the uterus;

FIG. 13b is a detail view of FIG. 13a, showing the distal end of the device according to the first embodiment of the present invention;

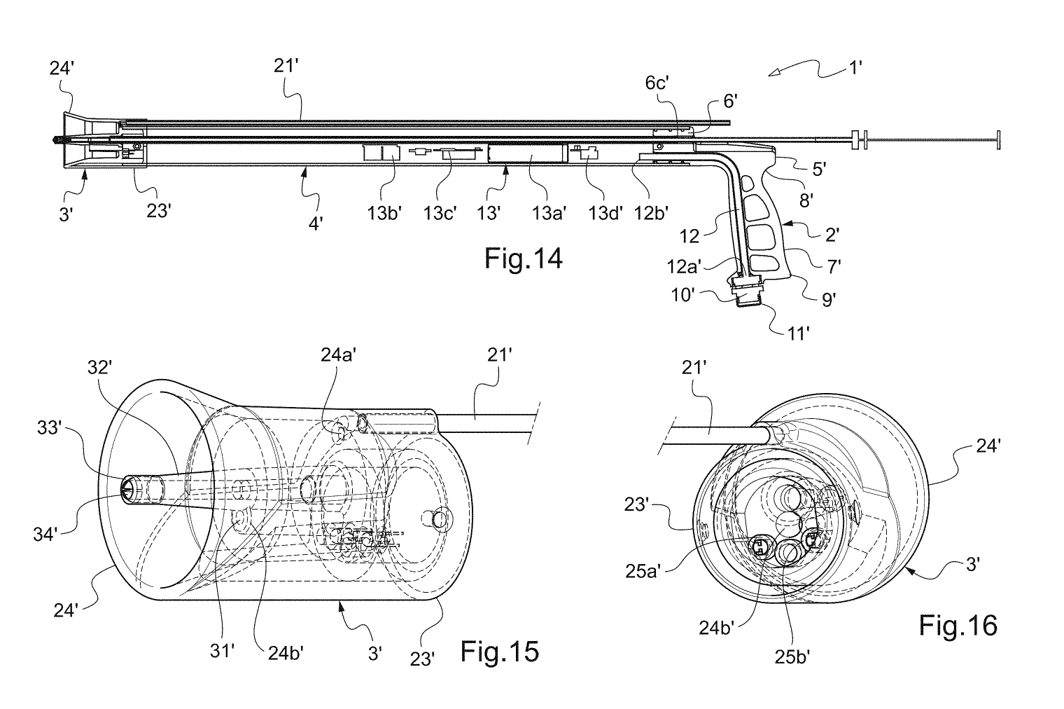

FIG. 14 is a side and sectional view of the device according to a second embodiment of the present invention;

FIGS. 15 and 16 are perspective views of the cup of the device according to the second embodiment of the present invention, the cup being seen from the front and the rear, respectively;

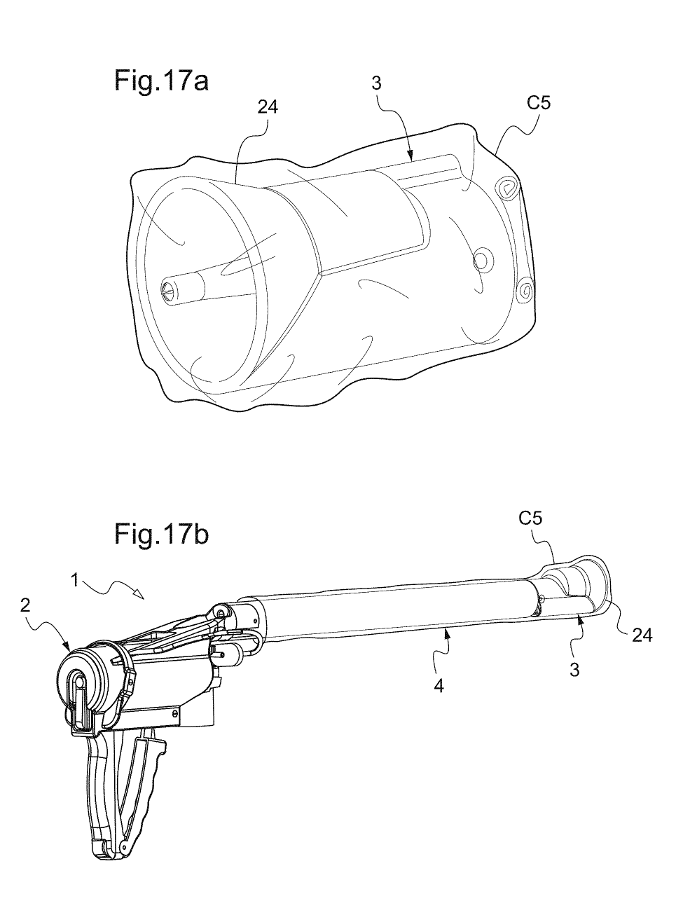

FIG. 17a is a perspective view of the cup of FIGS. 15 and 16, enveloped with a sanitary jacket;

FIG. 17b is a perspective view of the gun on which the jacket of FIG. 17a has been unrolled;

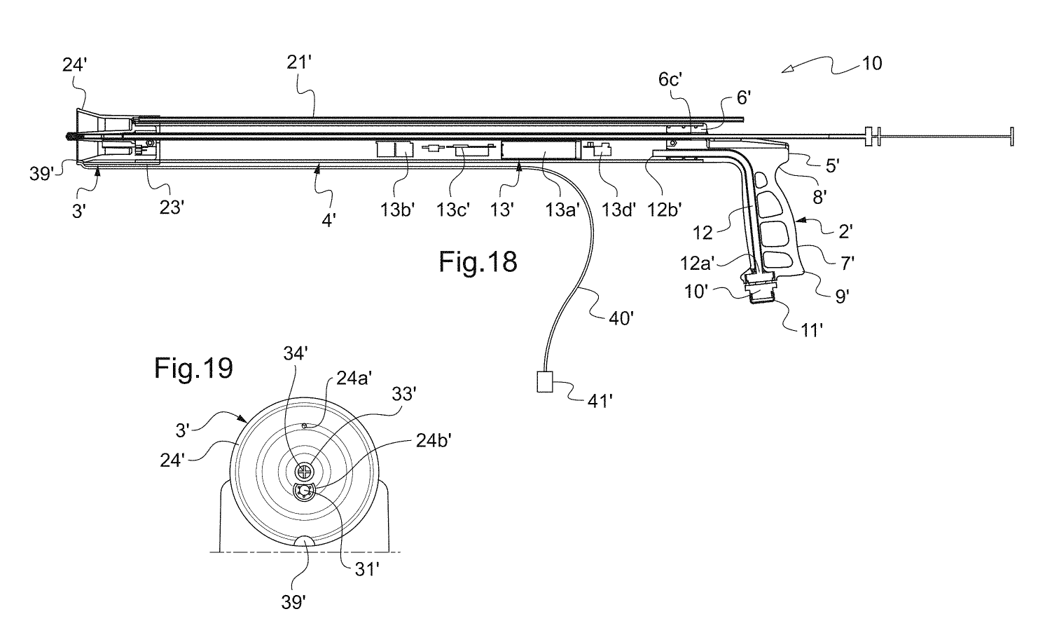

FIGS. 18 and 19 are views similar to respectively FIGS. 14 and 6, showing a variante of the device according to the second embodiment; and

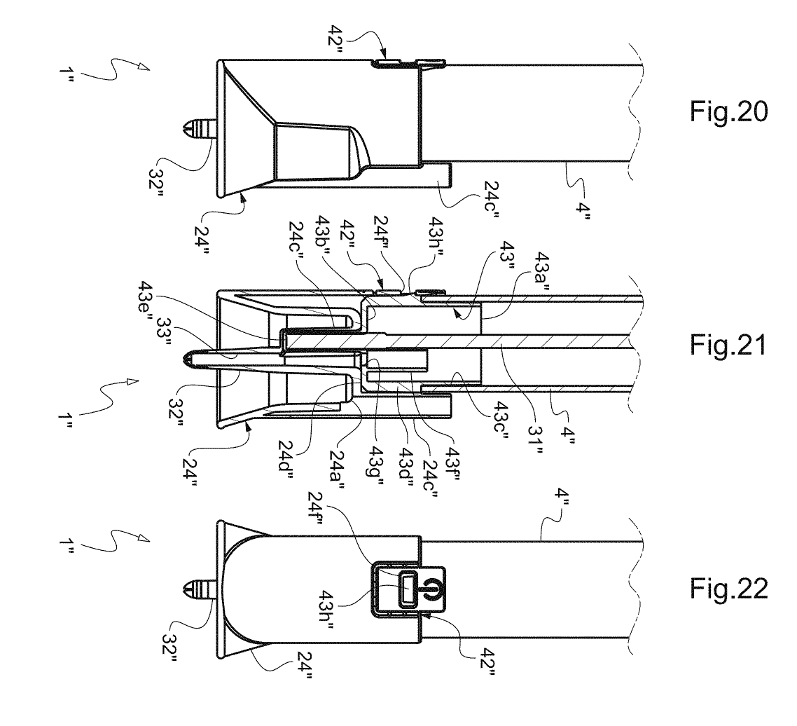

FIGS. 20, 21 and 22 are side, longitudinal sectional and top views, respectively, of the distal region of a device according to a third embodiment.

Referring to FIGS. 2 to 9, it can be seen that a device 1 for accessing, via the vagina, the uterus of a female livestock animal according to a particular embodiment of the present invention comprises a proximal body 2 and a distal part 3 connected by a tubular body 4, the device 1 having the general form of a gun.

The proximal body 2 comprises a hollow cylindrical part 5 having, in the upper distal region thereof, a projection 6 the distal end of which is cylindrical and has a diameter very slightly smaller than the inner diameter of the tubular body 4 so that said distal end is inserted into and secured to the tubular body 4.

The tubular body 4 is made of plastic material, such as PVC (polyvinyl chloride).

The proximal body 2 also comprises a handle 7, arranged diametrically opposite the projection 6, the handle 7 comprising a substantially straight fixed part 8, a first end 8a of which is fixed to the proximal end of the cylindrical part 5 and extends radially therefrom.

The handle 7 also comprises a substantially straight mobile part 9, a first end 9a of which is pivotably mounted on the second end 8b of the fixed part 8. The second end 9b of the mobile part 9 is secured to a first end 10a of a slide 10 which is substantially parallelepiped and slidably mounted in the lengthwise direction thereof in a groove 11 provided on the cylindrical body 5 and in the lengthwise direction thereof.

Thus, the second end 10b of the slide 10 is located more proximal than said first end 10a of the slide 10. The second end 10b has an elbow 12 having a straight part 13 extending radially toward the axis of revolution of the cylindrical part 5 at which it is secured to a plunger 14.

The plunger 14 comprises a head 15 and a rod 16 secured to the head 15 and directed toward the proximal side of the device 1. Thus, the end of the straight part 13 is fixed to the proximal end of the rod 16.

The plunger 14 is arranged in the hollow inner part of the cylindrical part 5 of the proximal body 2, and the inner space delimited by the inner surface of the cylindrical part 5 and by the plunger head 15 forms a chamber 17.

A spring (not shown) extends between the second end 9b of the movable portion 9 and the distal end of the groove 11, the spring returning the mobile part 9 to the initial position, as shown in FIG. 4, after the second end 9b has been moved to the proximal side of the device 1, as shown in FIG. 5.

The chamber 17, at the distal end thereof, is fluidically connected via a channel 18 to a reservoir 19 secured to the proximal body 2.

A first end of a suction duct 21 is connected to the distal part 3, the duct 21 passing inside the tubular body 4.

The reservoir 19 has an outlet orifice 20 connected to a first branch of a T-shaped quarter-turn valve 22, the second branch of the T shape being connected to the other end of the duct 21 and the third branch of the T shape being connected to an orifice opening outwards.

The valve 22 is actuatable between a first, so-called suction position, in which the reservoir 19 and the duct 21 are fluidically connected to one another and are not fluidically connected to the orifice opening outwards, thereby exerting a suction at the first end of the duct 21 upon the actuation of the plunger 14, and a second, so-called venting position, in which the reservoir 19, the duct 21 and the orifice opening outwards are fluidically connected, thereby returning the first end of the duct 21 to atmospheric pressure.

The distal part 3 comprises a body part 23, which is proximal, and a cup part 24, which is distal.

The distal part 3 can be, for example, made of transparent plastic material.

The cup part 24 is arranged with the open end thereof oriented toward the distal side of the device 1. In particular, the second end of the duct 21 is connected to an orifice 24a which is provided in the bottom of the cup part 24 and opens outwards.

The body part 23 is cylindrical and hollow, and is connected to the tubular body 4 via a joining piece 25.

The proximal end region of the joining piece 25 is cylindrical, has a diameter slightly smaller than that of the tubular body 4 and is inserted therein in a fixed manner.

Thus, the tubular body 4 extends between the projection 6 and the joining piece 25.

The distal end region of the joining piece 25 is cylindrical, has a diameter slightly smaller than that of the body part 23 and is surrounded by the latter.

The body part 23 and the joining piece 25 are connected by a pivot connection 26 located on a diameter of the joining piece 25, the connection 26 being substantially horizontal when the handle 7 of the proximal body 2 is directed downwards.

Moreover, when the handle 7 of the proximal body 2 is directed downwards, it can be seen in particular in FIG. 10 that the body part 23 surrounds the junction piece 25 only on the upper part thereof. A notch 23a is indeed provided on the body part 23. This notch 23a allows the distal part 3, when it pivots about the pivot connection 26, as shown in FIG. 5, not to be blocked by the joining piece 25 and thus to be able to be freely tilted.

The joining piece 25 comprises four orifices 25a, 25b, 25c, 25d. The duct 21 passes through the orifice 25a, the elements passing through the other orifices will be detailed later in the description.

A spring 27 connects the body part 23 and the joining piece 25 and is arranged on one side of an imaginary plane to which the axis of the pivot connection 26 and the longitudinal axis of the tubular body 4 belong.

On the other side of said plane, a first end of a wire 28 is secured to the body part 23 by means of a blocking piece 29, secured to the wire 28. The blocking piece 29 is force-fitted in a cavity (not shown) provided inside the body part 23.

The other end of the wire 28 is fixed in the upper part of a yoke 30 surrounding the proximal body 2, and the lower part of which is pivotably mounted on the outer walls of the part of the proximal body 2 defining the groove 11.

The wire 28 passes through an orifice 6a of the projection 6 of the proximal body 2, through the tubular body 4 and through the orifice 25b of the joining piece 25.

The device 1 also comprises a camera 31 of the endoscope type, with an integrated lighting source, the objective of which is flush with an orifice 24b provided in the bottom of the cup, and the other end of which is connected to display means (not shown), the camera passing through the orifice 25c of the junction piece 25, through the tubular body 4 and through an orifice 6b of the projection 6 of the proximal body 2, to be finally connected to a viewing device D which is schematically represented, such as a monitor, a computer, a tablet computer, a smartphone, etc.

The orifice 25c is provided at the center of the joining piece 25.

The cup part 24 also comprises a projection 32, provided at the center of the bottom of the cup part 24 and directed toward the distal side thereof, a through hole 33 extending from one end of the projection 32 to the other and opening on the outside at the top of the latter.

The through hole 33 is aligned with the hole 25d of the joining piece 25, the hole 25d being provided on the same side of the imaginary plane intersecting the piece 25 as the spring 27, and is located on the diameter perpendicular to the axis of the pivot connection 26.

In other words, the orifice 25d is just above the orifice 25c, when the gun is held with the handle 7 directed downwards.

Thus, the axis of revolution of the cup part 24 is slightly offset upward with respect to that of the body part 23.

The orifice 25d is also aligned with an orifice 6c of the projection 6, which forms a rectilinear path for the passage of a transfer or sampling member, the use of which will be described later in the description.

The through hole 33 is closed by a slit cap 34. The cap 34 is made of elastomeric material, has an ogive shape and has a cross slit, the centre of which corresponds to the apex of the ogive. It avoids interactions between the transfer or sampling member and the outside when said member does not project through the through hole 33.

The cap 34 is adapted to allow said member to pass through the slits.

The device 1 also comprises a plug 35, made of rubber-like material, removably inserted into the cup part 24. The plug 35 comprises a hole 36 through which a thread 37 passes, the thread 37 making it possible, by pulling on the latter, to dislodge the plug 35 from the cup part 24.

The plug 35 has a rounded shape at the distal end thereof, and the diameter thereof at the base is substantially the same as that of the cup part 24.

Thus, the assembly formed by the plug 35 and the cup part 24 does not have any angled edge, and thus does not pose a risk of injury to the animal, and has a shape facilitating the passage of the device 1 through the folds of the vagina of the animal.

As shown in FIGS. 10 and 11, in use, an operator places a transfer or sampling member 38 in the passage of the device 1 provided for this purpose, until the distal end of said member is located in the projection 32 of the cup 24, in the vicinity of the slit cap 34.

The device 1 is then in the initial position, as shown in FIG. 11, with the distal part 3 aligned with the tubular body 4, and with the plug 35 present.

The operator then inserts the device 1, by the distal end thereof, into the vagina V of a female livestock animal until the plug 35 comes into contact with the uterine cervix Cu of the animal.

The plug 35 is then removed by the operator, by pulling on the thread 37 that the operator will have taken care to pass along the device 1 to the outside.

Once the plug 35 is removed, the operator can then view the uterine cervix Cu with the camera 31, and align the projection 32 with the cervix Cu. It is emphasized here that the length of the projection 32 is greater than the depth of the cup 24; in other words, the projection 32 protrudes from the free peripheral edge of the cup 24 such that the free end of the projection 32 enters first into the cervix Cu and makes it possible to correctly centre the cup 24 relative to the cervix Cu.

It is possible for the operator to tilt the distal part 3, by actuating the yoke 30, if the configuration of the organs of the bovine requires it.

Thus, as can be seen in FIG. 4, when the upper part of the yoke 30 is moved toward the proximal side of the device 1, pulling is exerted on the wire 30, which has the effect of pivoting the distal part 3 downwards from its initial position, i.e. aligned with the tubular body 4.

The operator can thus more easily align the projection 32 with the uterine cervix Cu.

Once the uterine cervix Cu and the projection 32 are generally aligned, the operator places the cup part 24 against the fornix of the cervix Cu, and actuates the handle 9 in order to move the plunger 14 and create a negative pressure in the chamber 17.

This negative pressure, when the quarter-turn valve 22 is in the first position, creates a suction of the uterine cervix Cu into the cup part 24, and thus secures the cup part 24 to uterine cervix Cu so as to facilitate the passage of the transfer or sampling member 38. In particular, the operator can move the device 1 in order to align the rings A of the uterine cervix Cu.

Once the cup part 24 is secured to the uterine cervix Cu, it is no longer necessary to orient the distal part 3 of the device 1. Thus, when the force acting on the yoke 30 is released, the spring 27 exerts a force that returns the distal part 3 to its initial position, and by pulling on the wire 28, also replaces the yoke 30 in its initial position.

During the suction, secretions of the animal may be sucked by the device 1. These secretions pass through the suction orifice 24a, then by the suction duct 21 and the quarter-turn valve 22, and then drop by gravity into the collection container 19. The channel 18 connecting the container 19 to the chamber 17 is located in the upper part of the container 19, so that secretions cannot go into the chamber 17, thereby avoiding poor suction performance.

Maintaining suction allows an operator to manipulate the uterine cervix with the device. Another, secondary; advantage is that it prevents vaginal secretions of the animal from penetrating into the uterus U, thereby avoiding contamination of the uterus U by bacteria that can be present in these substances.

The operator then pushes the transfer and sampling member 38 to pass the cap 34, until the distal end of said member is in the uterus. This position is shown in FIGS. 13a and 13b.

It can be seen in particular in FIG. 13b that the bell shape of the cup part 24 is provided so as to conform to the shape of the uterine cervix Cu, thereby ensuring the securing of the cup part 24 with the uterine cervix Cu.

Once the transfer or sampling operation has been carried out, the operator removes the transfer or sampling member from the device 1. The operatore then actuates the quarter-turn valve 22 to place it in the second position, in which the reservoir 19 and the duct 21 are then set to atmospheric pressure, thereby separating the cup part 24 from the uterine cervix Cu, and thus allows the removal of the device 1 from the bovine.

The device 1 according to the present invention therefore allows an operator alone, in particular the breeder, to access, in a simple manner, via the vagina, the uterus of a female livestock animal for the purpose of a transfer of material or substance with a reproductive, therapeutic or diagnostic purpose, or for the purpose of a sampling from the uterus, and to carry out said transfer or sampling.

Referring to FIGS. 14 to 16, it can be seen a device 1′ according to a second embodiment of the present invention, which substantially comprises the same elements as the device 1 described above. Thus, the device 1′ comprises a proximal body 2′ and a distal part 3′ connected by a tubular body 4′, the device 1′ having the general form of a gun.

The proximal body 2′ comprises a substantially half-cylinder body part 5′ having, in the upper distal region thereof, a projection 6′, the distal end region of which is cylindrical and has a diameter slightly smaller than the inner diameter of the tubular body 4′, said distal end region being inserted into and secured to the tubular body 4′.

As in the first embodiment, the tubular body 4′ is made of a plastic material, such as PVC (polyvinyl chloride).

The proximal body 2′ also comprises a handle 7′, arranged diametrically opposite the projection 6′, the handle 7′ having a first end 8′ fixed to the proximal end of the body part 5′ and extending radially therefrom.

The handle 7′ also comprises, at the second end 9′ thereof opposite the first end 8′, a connector 10′ of the USB port type, which can be closed by a plug 11′.

The connector 10′ is connected to the first end 12a′ of a harness 12′. The other end 12b′ of the harness 12′ is connected to an electronic wireless transmission assembly 13′, said assembly 13′ comprising a battery 13a′ and electronic boards 13b′, 13c′, 13d′ comprising wireless data transmission chips.

The connector 10′ allows the device 1′ to be connected to a power source, which recharges the battery 13a′ when the latter is empty.

A first end of a suction duct 21′ is connected to suction means (not shown), such as a manual vacuum pump, the other end of the duct 21′ being connected to the distal part 3′, the duct 21′ extending along the tubular body 4′, on the outside thereof.

The distal part 3′ comprises a body part 23′, which is proximal, and a cup part 24′, which is distal.

The cup part 24′ is arranged with the open end thereof oriented toward the distal side of the device 1′. In particular, the other end of the duct 21′ is connected to an orifice 24a′ which is provided in the bottom of the cup part 24′ and opens outwards.

The body part 23′ is cylindrical and hollow, and surrounds the tubular body 4′.

The device 1′ also comprises a camera 31′, the objective of which is flush with an orifice 24b′ provided in the bottom of the cup part 24′, and the body of which is connected, at the other end thereof, to the wireless data transmission assembly 13′, the body of the camera 31′ passing inside the tubular body 4′.

The wireless data transmission assembly 13′ is configured to receive the images recorded by the camera 31′ and to transmit the latter in a wireless manner, preferably by WiFi, to a remote viewing device such as a computer, using the electronic boards 13b′, 13c′, 13d′.

The cup part 24′ allows at least a part of the the light to pass. It may thus be transparent or translucent for example. Polyethylene or polypropylene, for example, could be used for the distal part 3′.

The cup part 24′ comprises, in the proximal part thereof, on the side opposite the bottom of the cup part 24′ and which faces outside, light-emitting diodes (LEDs) 25a′ and 25b′ mounted in cylindrical housings formed by the cup part 24′. The LEDs 25a′ and 25b′ have an illumination direction oriented toward the distal end of the cup part 24′.

The LEDs 25a′ and 25b′ are powered by the battery 13a′ of the device 1′, and illuminates, due to the transparency of the material forming the cup part 24, the uterine cervix of the animal. Thus, the light provided by the LEDs enables the camera 31′ to capture images of the surroundings ahead of the cup 24′ for an easier positioning of the device 1′ against the uterine cervix of the animal and an easier alignment of the rings of the uterine cervix.

The cup part 24′ also comprises a projection 32′, provided at the centre of the bottom of the cup 24′ and directed toward the distal side thereof, the projection 32′ comprising a through hole 33′ closed by a slit cap 34′, in a manner similar to the first embodiment. As in the first embodiment, the axis of revolution of the cup part 24′ is slightly offset upward with respect to that of the body part 23′.

Although not shown, the device 1′ may also comprise a plug similar to the plug 35, with an associated thread.

The device 1′ according to the second embodiment is used in a manner similar to the device 1, except that the suction is not obtained by a trigger type mechanism, but by actuating separate suction means (manual vacuum pump), and that the device 1′ is provided with electronics enabling it to wirelessly transmit the images recorded by the camera 31′.

Referring now to FIGS. 17a and 17b, it can be seen that there is shown a cup 24 enveloped by a sanitary jacket CS, which protects it from the outside. The sanitary jacket CS is arranged rolled up on the cup 24, as shown in FIG. 17a, and after the cup 24 has been fitted onto the body assembly 4, the sanitary jacket CS is unrolled on the body assembly 4, so as to protect the distal part of the device 1, as shown in FIG. 17b. The sanitary jacket CS can also be used with the cup 24′ according to the first embodiment.

Referring now to FIGS. 18 and 19, it can be seen a variant of the device 1′ according to the second embodiment, which differs from the latter only in that it comprises means for sucking up possible phelgms which could obstruct the field of view of the camera 31′.

These means comprise a phlegm suction through hole 39′ which opens, at the distal end thereof, inside the cup 24′, and, at the proximal end thereof, on the outside of the cup 24′, for example at a connector-forming tubular part, to which the distal end of a suction duct 40′ extending along the body 4′ can be sealingly connected, for example by force-fitting, while the proximal end of the suction duct 40′ is connected to suction means 41′ represented very schematically, and which may for example be a manually actuated, or foot actuated, vacuum pump.

It is easily understood that actuating the suction means sucks elements which would be preset in the cup, and in particular phlegms. For this purpose, the phlegm suction through hole 39′ will be dimensioned so as to be able to suck phlegms (i.e. it will be sufficiently large).

In practice, when the operator no longer sees the inside of the animal during the insertion of the device 1′, or during the material or substance transfer or sampling operation, because phlegms obstruct the field of view of the camera 31′, the operator just needs to actuate the phlegm suction means 41′, which will then suck said phlegms, thereby clearing the field of view of the camera 31′.

In the example shown, the orifice 24a′ and the hole 39′ are diametrically opposite to each other. Of course, the present invention is not limited to this positioning, and the orifice 24a′ and the hole 39′ could be provided in any position. For example, it will be advantageous to provide them both in proximity to each other, on the lower side of the cup 24′ when looking at FIGS. 18 and 19, so that the suction ducts 21′ and 40′ both extend close to each other.

Referring to FIGS. 20 to 22, it can be seen that there is shown the distal region of a device 1″ according to a third embodiment, which has, comparing to other embodiments, the specific feature of having a cup 24″ connected to the tubular body 4″ in a removable manner by reversible snap-fit attachment means 42″.

As in the above embodiments, the cup 24″ comprises a projection 32″ having a through hole 33″ to allow the transfer of material or substance, or the sampling, as well as an orifice 24a″ which is configured to be connected to a suction duct (not shown) which is connected to a connector-forming outer part 24c″ equipping the cup 24″, on the same principle as the suction duct 21′ of the second embodiment.

The cup 24″ is distinguishable in that it has, on the proximal side, a cylindrical recess 24d″ configured to allow the cup 24″ to be mounted on an attachment piece 43″.

The attachment piece 43″ is in the form of a hollow cylindrical piece opened at a proximal end 43a″ and the distal end of which is formed by a bottom 43b″. The attachment piece 43″ has a proximal region 43c″ having a first outer diameter dimensioned for allowing it to be received in the tubular body 4″, for example with force-fitting, while the distal region 43d″ of the attachment piece 43″ has a second diameter greater than the first diameter and dimensioned to be received in the cylindrical recess 24d″ of the cup 24″. The attachment piece 43″ thus has a shoulder between the proximal region 43c″ and the distal region 43d″, and said shoulder will abut against the distal end of the tubular body 4″. In other words, the attachment piece 43″ is interposed between the tubular body 4″ and the cup 24″.

A hollow tubular projection 43e″ extends from the bottom 43b″ of the attachment piece 43″, along a longitudinal direction parallel to that of the attachment piece 43″. The camera 31″, here of the endoscopic type, extends into the inner space of the hollow projection 43e″, to the bottom thereof. The cup 24″ has a second cylindrical recess 24e″ extending from the bottom of the cylindrical recess 24d″ and dimensioned so that the hollow projection 43e″ extends therein when the cup 24″ is mounted on the attachment piece 43″.

Furthermore, a tubular part 43f″ extends from the bottom 43b″, parallel to the longitudinal direction of the attachment piece 43″ and adjacent to the entry opening of the hollow protrusion 43e″. The tubular portion 43f″ is open at the proximal end thereof and opens onto a through hole 43g″ which is provided in the bottom 43b″ in such a position that it faces the through hole 33″ of the projection 32″ when the cup 24″ is mounted on the attachment piece 43″, so as to allow the passage of a device for transferring material or substance with a reproductive, therapeutic or diagnostic purpose. In other words, the tubular portion 43f″ and the inner space of the attachment piece 43″ forms a through passage in communication with the through passage of the tubular body 4″.

According to an advantageous variant, the cup 24″ and the attachment piece 43″ are both transparent or translucent and, in the case of a camera not of the endoscopic type, a lighting source, such as at least one light-emitting diode, can be arranged in the inner space of the cylindrical part 43″ so as to provide the lighting for the camera, while remaining protected from the external surroundings.

The cup 24″ is connected to the attachment piece 43″ in a removable manner by reversible snap-fit, as follows. The cup 24″ has an opening 24f″, here rectangular, provided in the wall of the cylindrical recess 24d″, and a tooth 43h″ is formed by a slight bulge on the outer surface of the distal region 43d″ of the attachment piece 43″. As can be seen in FIGS. 21 and 22, the tooth 43f″ is formed by a rectangular face slightly inclined in a direction parallel to the longitudinal direction of the attachment piece 43″, apart from the outer surface of the distal region 43d″ as it approaches the proximal region 43c″, to terminate into a vertical face connecting said inclined face to the outer surface of the distal region 43d″. The opening 24f″ and the tooth 43h″ are positioned such that the tooth 43h″ extends into the opening 24f″ when the cup 24″ is in the position of use (mounted on the attachment piece 43″), with an edge of the opening 24f″ abutting against the vertical face of the tooth 43h″.

It will easily be understood that when the operator mounts the cup 24″ on the attachment piece 43″, the latter will be slightly deformed inwardly, in the region thereof having the tooth 43h″, until the bottom of the recess 24d″ of the cup 24″ comes into contact with the bottom 43b″ of the attachment piece 43″, at which time the tooth 43h″ will return elastically to its undeformed position and thus will come into abutment against an edge of the opening 24f″, preventing the cup 24″ from being translated in the proximal-distal direction. The cup 24″ is thus firmly mounted in position for the use.

After the operations associated with the transfer of material or substance or with a sampling are completed, and the device 1″ has been removed from the animal, the operator can disassembly the cup 24″ with one hand by simply pressing on the tooth 43h″ so that it is moved from the opening 24f″, while pulling on the cup 24″ in order to separate it from the attachment piece 43″.

Of course, the attachment piece 43″ could be made of a material allowing such a deformation.

Removing a cup 24″ that has just been used and replacing it with a new cup 24″ is therefore particularly fast and simple. In other words, the cups 24″ become consumables.

Obviously, other removable attachment means between the cup and the attachment piece could be provided.

In addition, the principle of an attachment piece that, on one hand, is secured to the body assembly and, on the other hand, removably connected to the cup, could be applied to the first embodiment in which a joining piece is provided to allow tilting of the cup. In such a case, the proximal region of the attachment piece could be formed similarly to body part 23 of the cup 24 (FIGS. 4 and 5), the connecting means between the attachment piece and the cup remaining unchanged.

It is understood that the above embodiments of the present invention have been given for information and non-limiting purposes, and that modifications could be made thereto without departing from the scope of the present invention.

1-17. (canceled)

18. A device for accessing, via the vagina, the uterus of a female livestock animal for one of the purpose of a transfer of material or substance with a reproductive, therapeutic or diagnostic purpose, and the purpose of a sampling from the uterus, and for carrying out said transfer or sampling, the device comprising a body assembly, which is integral, at a proximal end thereof, with grasping means for an operator and has a distal end, a first through passage extending through the device and opening out at the proximal and distal ends, so as to establish an access route between an inside and an outside of the animal, so as to allow the transfer of material or substance with a reproductive, therapeutic or diagnostic purpose, or the sampling,
wherein the body assembly is substantially straight and rigid so as to allow the body assembly to be inserted into the vagina of the animal, and wherein is provided, at the distal end of the body assembly, a cup, an opening of said cup being oriented away from the proximal end of the body assembly, a central projection extending from a bottom of the cup and having a through hole, an end of said through hole being in communication with the first through passage of the body assembly, the cup also having an orifice in communication with a distal end of a second through passage provided in the body assembly and a proximal end of said second through passage being configured for connection to suction means which are configured to create in the cup a negative pressure that presses the cup against the fornix of the animal, the first through passage then constituting an access route to the uterus from the outside of the animal, the cup also comprising a second hole adapted to receive viewing means having a field of view oriented in a distal direction of the device to allow an operator to view the surroundings ahead of the cup.

19. The device according to claim 18, wherein the viewing means comprise a camera having an objective received in the second hole, a lighting source and transmission means for transmitting images captured by the camera to a viewing device having a screen allowing the operator to observe said captured images.

20. The device according to claim 19, wherein the camera is an endoscopic camera in which the lighting source is integrated, said transmission means being formed by the body of the endoscopic camera, said body extending along the body assembly to the proximal end region of the body assembly, where the endoscopic camera is connected to the viewing device.

21. The device according to claim 19, wherein the transmission means comprises at least one electronic circuit housed in the body assembly and connected to the camera, the at least one electronic circuit being configured to wirelessly transmit to the viewing device the images captured by the camera.

22. The device according to claim 21, wherein the cup is transparent and comprises, on a rear part thereof, light-emitting diodes arranged so as to illuminate in the distal direction of the device, a light beam of the diodes going through the transparent cup and the diodes thus constituting the lighting source for the camera.

23. The device according to claim 19, wherein the cup is removably connected to the body assembly.

24. The device according to claim 23, wherein the cup is removably connected to the body assembly by means of the quick-release attachment type.

25. The device according to claim 23, wherein it comprises an attachment piece, which is integral with the body assembly and comprises a distal region extending from the body assembly, the cup having, on the proximal side, a recess configured to receive said distal region, either one, or both, of the cup and said distal region having the means of the quick-releasing attachment type, the attachment piece further having a through passage in communication with the first through passage of the body assembly and with the through hole of the projection of the cup when the cup is attached to the attachment piece.

26. The device according to claim 25, wherein the viewing means comprise a camera having an objective received in the second hole, a lighting source and transmission means for transmitting images captured by the camera to a viewing device having a screen allowing the operator to observe said captured images, and wherein the transmission means comprises at least one electronic circuit housed in the body assembly and connected to the camera, the at least one electronic circuit being configured so as to wirelessly transmit to the viewing device the images captured by the camera, the attachment piece and the cup being transparent, a lighting source being mounted inside the attachment piece so as to illuminate in the distal direction of the device.

27. The device according to claim 18, wherein the body assembly is of cylindrical shape and comprises a body part which is integral with the grasping means.

28. The device according to claim 18, wherein the orifice of the projection of the cup is closed by a cap, made of elastomeric material, which is integral with the projection, the cap protecting the transfer or sampling member with respect to potential secretions from the animal after removal of a plug selectively closing the distal end of the cup, the cap having a central cross slit allowing a transfer or sampling member to go through the cap when said member is pushed by an operator, and thus to go beyond the projection.

29. The device according to claim 18, wherein the grasping means comprise a handle and a body, the grasping means having, in combination with the body assembly, the shape of a gun.

30. The device according to claim 27, wherein the suction means comprise a manual pump having a plunger and a chamber, a part of the handle of the grasping means being pivotably mounted with respect to the body of the grasping means and being connected to the plunger, whereby a movement of the handle actuates the plunger and creates a suction in the chamber.

31. The device according to claim 18, wherein the body assembly is of a cylindrical shape, and comprises a body part which is integral with the grasping means and a head part constituting the distal end of the body assembly, the head part being connected to the body part by a pivot connection along a diameter of the body assembly, the device further comprising actuating means which orient the head part between a so-called straight position in which the body part and the head part are aligned on a same straight line, and a so-called tilted position in which the body part and the head part form a non-zero angle to one another.

32. The device according to claim 31, wherein the actuating means orienting the head part comprise a yoke, a wire and a spring, the yoke being pivotably mounted on and with respect to the grasping means, the wire connecting the yoke and a first connection point provided in the head part, and the spring connecting the head part and a second point provided in the body part, the first and second points being located on either side of an imaginary plane which includes the pivot connection and divides the body part longitudinally into two equal parts, such that the head part is brought to the titled position when the yoke is pivoted toward the proximal end of the device, by pulling on the wire, and returns to the straight position under the action of the spring when the yoke is released.

33. The device according to claim 18, wherein the cup comprises a phlegm suction through hole, wherein a distal end of said phlegm suction through open opens on the opening side of the cup and a proximal end of said phlegm suction through hole is configured to be connected to a distal end of a suction duct, the proximal end of said suction duct being configured to be connected to phlegm suction means which are in turn configured to create a negative pressure for sucking the potential phlegm from the opening of the cup to the outside of the device via the phlegm suction through hole and the suction duct.

34. A cup for a device as defined in claim 18, wherein an opening of the cup is, in use, oriented away from the proximal end of the body assembly of said device, a central projection extending from a bottom of the cup and having a through hole, an end of said through hole being in communication with the first through passage of the body assembly, the cup further having an orifice in communication with a distal end of a second through passage provided in the body assembly, the cup also comprising a second hole adapted to receive viewing means having a field of view oriented in the distal direction of the device to allow an operator to view the surroundings ahead of the cup.

35. The cup according to claim 34, wherein the cup also comprises a sanitary jacked, which is substantially tubular and closed at a distal end thereof, rolled up on itself at a proximal end thereof, and arranged so as to envelop the cup and be able to be unrolled on the body assembly once the cup has been attached to said device.
